(19) United States
(12) Patent Application Publication
(10) Pub. No.: US 2026/0335853 A1
(43) Pub. Date: Oct. 29, 2026

(54) CHEMICAL REACTOR WITH A HEAT EXCHANGER

(71) Applicant: CASALE SA, Lugano (CH)

(72) Inventors: Nicola PANZERI, Breganzona (CH); Andrea BERETTI, Rovellasca (CO) (IT); Tiziano MAFFEI, Como (IT)

(21) Appl. No.: 19/122,332

(22) PCT Filed: Sep. 14, 2023
(86) PCT No.: PCT/EP2023/075341

§ 371 Date: Apr. 18, 2025

(30) Foreign Application Priority Data
Oct. 27, 2022 (EP) 22204219.4

(51) Int. Cl. B01J 8/02; C01C 1/04; C07C 29/00

(52) U.S. Cl. CPC B01J 8/0285; B01J 8/0257; B01J 8/0278; C01C 1/04; C07C 29/00; B01J 2208/00115; B01J 2208/00884

(57) ABSTRACT

A chemical reactor, comprising a catalytic bed and a heat exchanger arranged to transfer heat from a first fluid, which is effluent of said catalytic bed, to a second fluid, which is a cooling medium or a heating medium, wherein said heat exchanger comprises a structure made by additive manufacturing, said structure being a triply periodic minimal TPMS structure having a first side and a second side that can be traversed by heat exchange fluids, and said heat exchanger comprises a set of boundary elements arranged to feed and collect said first fluid and second fluid to/from channels of said first side and channels of said second side of the structure.

### FIELD OF APPLICATION

The invention is in the field of catalytic reactors with a heat exchanger inside.

### PRIOR ART

Many chemical reactions of considerable industrial interest require a reactor including one or more heat exchangers. Said heat exchanger(s) may remove the reaction heat in case of exothermic reaction or to provide the reaction heat in case of endothermic reaction.

A noticeable example is represented by the synthesis of ammonia or the synthesis of methanol, which are strongly exothermic. Such reactions are performed in reactors comprising one or more catalytic bed(s). Due to exothermicity, the effluent of the catalytic beds is typically at a high temperature and there is an interest to remove heat from such effluent to preheat another process stream or to produce steam, as well as to suitably cool the effluent for a subsequent reaction step.

The most common heat exchanger for use in chemical reactors is the shell-and-tube heat exchanger. In a shell-and-tube heat exchanger, the tubes are traversed by a first fluid and a second fluid circulates around the tubes, confined by a shell. Conventionally, the inside of the tubes is called tube side and the space around the tubes is called shell side.

A notable example is represented by inter-bed heat exchangers of multi-bed ammonia reactors. In a multi-bed reactor, the conversion is usually performed in a plurality (such as two or more, generally three or four) catalytic beds arranged in series, so that the effluent of a bed is further reacted in the next bed. The inter-bed exchangers cool the effluent of one bed before it is admitted in the next bed. In a common case, the catalytic beds have an annular shape so that a shell-and-tube exchanger can be accommodated in the central cavity of the bed.

In most applications, the tubes have a considerable length which makes them subject to vibrations. Additionally, it is desirable that the gaseous flow in the shell side be perpendicular to the tubes, to maximize the heat exchange. To this purpose, a shell-and-tube heat exchanger comprises transversal baffles arranged to suppress vibrations and to increase vorticity in the shell side.

However, the conventional shell-and-tube heat exchangers with baffles still have limits. The heat exchange surface is relatively small compared to the overall size of the exchanger, such as diameter of the outer shell; the vorticity induced by the baffles is confined in a small region near the baffles; the cost and complication are increased by the baffles.

A chemical reactor comprising a catalytic bed and a heat exchanger is described in DE 10 2015 114201. A heat exchanger with a gyroid minimal surface is described in US 2020 0033070 or EP 4 033 193. US 2018 0187984 describes monolithic bi-continuous core (MBC) structures for heat exchangers. US 2022 0003503 describes systems and methods for periodic nodal surface-based reactors, distributors, contactors and heat exchangers.

### SUMMARY OF THE INVENTION

The invention aims to solve the above drawbacks of heat exchangers installed in chemical reactors. The invention aims to provide a novel chemical reactor which solves the above problems.

The present invention uses, as a heat exchange body in a chemical reactor, a triply periodic minimal surface TPMS structure made by additive manufacturing. Said structure has interesting properties for heat exchange, including periodicity and the ability to generate a diffuse vorticity. The TPMS structure can also be regarded as a TPMS lattice.

A TPMS structure inherently defines two sides, which can be regarded as first side and second side or, in a heat exchange application, as hot side and cold side. The term hot side denotes a side of the structure traversed by the fluid having a higher temperature (“hot fluid”) and cold side denotes a side traversed by the fluid at lower temperature (“cold fluid”).

In the invention, a suitable set of boundary elements distributes and collects the hot fluid and the cold fluid to/from a respective side of the TPMS structure. In the structure, the two sides are fully separate, which means the hot fluid and cold fluid do not come into contact and exchange heat indirectly through the structure.

Accordingly, the aims of the invention are reached with a chemical reactor according to claim 1. Preferred features are stated in the dependent claims.

The advantages of the invention are the following.

The heat exchanger can be realized without baffles and with channels of small size. Accordingly, the Reynolds number of the fluid crossing the channels increases, and the effect of high specific pressure drop is reduced by the shorter length of the exchanger. The thickness of the walls forming the channels of the heat exchanger can be minimized, which increases the heat transfer coefficient.

The additive manufacturing technique gives several advantages including fast and cost-effective production, and the possibility to print the heat exchanger on demand having a wide range of customization in terms of size and shape.

Further a cost-effective production can be obtained because the amount of material, for example of alloys, needed to make the heat exchanger is lower compared to traditional methods. The materials waste generated during the manufacturing process is also reduced.

Due to the higher heat transfer coefficient, the heat exchanger can be manufactured in a compact size. This is particularly advantageous because the volume occupied by the heat exchanger in the catalytic reactor can be reduced and the volume allocated to the catalytic bed can be increased.

The invention may be used, among others, in the field of plants that exploit renewable energy sources to produce one or more of reagents for the synthesis.

The invention provides a compact heat exchanger which may save typically 50% to 90% space compared to a conventional rod baffle heat exchanger.

Another advantage is associated with the construction of the TPMS heat exchanger which comprises boundary elements having a thin wall but able to withstand high external pressure forces and to prevent buckling phenomena A further and notable aspect of the invention addresses the problem of how to connect a TPMS lattice with a conventional collector, such as a pipe. This task is challenging due to the complex shape of the TPMS lattice. The invention addresses this problem by providing a connection portion formed together with the TPMS lattice and a transition region between the TPMS lattice and said connection portion, wherein the shape of the TPMS lattice is gradually modified to match the collector. The connection portion can be, advantageously, formed monolithically with the TPMS lattice. Accordingly, the invention provides a heat exchanger based on a TPMS lattice which can be seamlessly connected to conventional distribution or collection pipes.

Still another aspect of the invention concerns a parallel-connected heat exchanger made by additive manufacturing.

### DESCRIPTION OF THE INVENTION

The chemical reactor of the invention comprises a heat exchanger having a structure made by additive manufacturing (AM) which is a triply periodic minimal surface TPMS. The term TPMS denotes a non-intersecting 3D surface characterized by a zero value of mean curvature at each point of the surface. TPMS surfaces are defined with mathematical functions which are known in the art and for this reason they are no discussed in detail herein. The term TPMS lattice may also be used to denote said structure.

The TPMS structure (TPMS lattice) can be regarded as a portion of the internal volume of the chemical reactor which performs the function of a heat exchanger. The TPMS structure defines two sides adapted to be traversed by a hot medium and a cold medium, respectively, so it can actually work as a heat exchanger.

The TPMS structure can be described mathematically by a suitable equation. Preferably the TPMS structure is defined by an implicit function having the form:

$$G ( \overline{X} ) = 0$$

wherein X denotes a suitable set of parameters, including spatial coordinates and one or more parameters to define features of the TPMS structure, such as shape and thickness. For example, the above function can take the form:

$$G ( x , y , z , S , k ) = 0$$

wherein z, y, z are spatial coordinates; S is a set of one or more parameters describing the TMPS structure; k is a parameter defining the thickness of the structure.

In addition, the shape of the heat exchanger can be modelled by combining the TPMS structure with other shapes using operators such as boolean operators. For example, a hole passing through the TPMS structure can be easily defined by subtracting a cylinder from the TPMS structure. More formally, assuming A denotes a TPMS in a given region of space and B denotes a cylindrical pipe crossing the TPMS, the operation (A NOT B) will produce a TPMS with a passing-through hole.

The mathematical description of the TPMS structure provides an input for the design of the structure. A suitable reference concerning the mathematical description of TPMS and a way of modelling them by means of marching cube method can be found in:
Manuscripta Mathematica, Volume 64, 1989, Springer;
Marching cubes: a high resolution 3d surface construction algorithm, William E. Lorensen and Harvey E. Cline, Computer Graphics Vol. 21, No. 4, July 1987, 163-169;
Surface curvature in triply-periodic minimal surface architectures as a distinct design parameter in preparing advanced tissue engineering scaffold, Sébastien Blanquer, Maike Werner, Markus Hannula, Shahriar Sharifi, Guillaume Lajoinie, David Eglin, Jari Hyttinen, André Poot, Dirk Grijpma, IOP Publishing, 2017, 9 (2), pp. 025001. 10.1088/1758-5090/aa6553 hal-02336718;

The TPMS structure of the heat exchanger has a first side and a second side wherein said first side and said second side define respectively a first path and a second path that can be traversed by a heat exchange medium and/or by a reagent gas mixture. The first side can be crossed for instance by a coolant medium such a water and the second side can be crossed for instance by a reactant gas mixture.

The first path is separated from said second path, so that the heat exchange medium that travels the TPMS structure through the first path does not come into direct contact with the medium crossing the second path.

The heat exchanger also comprises a set of boundary elements which is configured so that said a first medium is distributed into and collected from the first side only and a second medium is distributed into and collected from the second side only. For example, the first medium is an effluent of a catalytic bed and the second medium is a heating medium or a cooling medium. In a reactor for exothermic reactions, the first medium is a hot effluent of the catalytic bed, from which heat is removed, and the second medium is a cooling medium, such as water, steam, or a process gas to be pre-heated.

The set of boundary elements is arranged so that the effluent of the catalytic bed can traverse the TPMS structure through the first path and the heating or cooling medium can traverse said structure through the second path, to obtain indirect transfer of heat from the effluent to the cooling medium or from the heating medium to the effluent, according to the enthalpy of the chemical reaction, namely the reaction being exothermic or endothermic.

The provision of boundary elements solves the challenging problem of how to distribute and collect heat exchange fluids to/from a TPMS structure.

According to an interesting application, the set of boundary elements is also made by additive manufacturing. Advantageously a high degree of flexibility in the design of the heat exchanger can be obtained.

The set of boundary elements can include one or more surface elements having a gas-permeable pattern arranged to match a pattern of inputs or outputs in the TPMS structure. The set of boundary elements may include one or more cylindrical shells in certain embodiments. In certain embodiments the boundary elements may have different shapes such as plates or rings, depending on the geometry of the heat exchanger.

The TPMS structure of the heat exchanger can have any suitable shape. In an embodiment of the invention, the TPMS structure has an annular shape and one side of said structure is traversed radially and said set of boundary elements includes an inner cylindrical shell around an inner surface of the bed and/or an outer cylindrical shell around an outer portion of the bed, to distribute and/or collect the effluent or the heating or cooling medium to/from said radially traversed side of the TPMS structure.

The catalytic bed in a preferred embodiment has an annular shape and is arranged around the TPMS structure. Accordingly said outer cylindrical shell may acts as a boundary element for the TPMS structure and as a retainer wall of catalyst granules which form the catalytic bed.

In an embodiment, the TPMS structure, transition and boundary elements are placed under the catalytic bed.

In a particular configuration of the previous embodiment, the TPMS structure heat exchanger is arranged to support the weight of the catalytic bed.

In an embodiment, the catalytic bed and the TPMS structure have an annular shape and the TMPS structure is arranged coaxially outside or inside the catalytic bed.

In an embodiment, the TPMS structure has an annular shape and is traversed radially by one fluid and axially by another fluid. The boundary element may include preferably a first ring-shaped boundary element placed above the structure and a second ring-shaped element below the structure, to distribute and collect the fluid which traverses the structure in the axial direction.

In another embodiment, the TPMS structure has an annular shape, and the set of boundary elements includes a first surface extending over a portion of an inner or outer surface of the TPMS structure, and a second surface arranged around a different portion of said surface, so that one fluid enter the TPMS structure and leaves the TPMS structure through different portions of said surface.

Preferably said first side and said second side include multiple channels in the TPMS structure. The channels of the heat exchanger can have a nearly-circular cross-section. Preferably the cross-section has a hydraulic diameter in the range of 2 mm to 20 mm and more preferably of 5 mm to 15 mm.

The surface of the TPMS structure is preferably selected from the group consisting of Gyroid, Schwarz minimal surface, and Neovius surface. In embodiments with a Schwarz minimal surface, said surface is preferably a Schwarz-P surface or a Schwarz-D (diamond) surface.

The TPMS structure and/or said set of boundary elements can be made by any suitable materials able to withstand the operating conditions of a synthesis reactor. Preferably said material is Inconel, particularly preferably Inconel 625 or Inconel 718 or a combination of them.

Any additive manufacturing technique can be used for manufacturing the heat exchanger and the set of boundary elements, provided the selected technique is suitable for the making of the structure of the heat exchanger or of the boundary elements. Preferably said technique is one of the following: powder bed fusion, such as preferably any of direct metal laser sintering, electron beam melting, selective heat sintering, selective laser melting and selective laser sintering; binder jetting; stereolithography; fused deposition modelling; digital light process; multi jet fusion; polyjet; direct energy deposition.

The internal surface of a heat exchanger according to the invention follows a TPMS surface. As stated above, the TPMS surface is described mathematically whereas the physical (real) surface, unlike a mathematical surface, has a non-zero thickness. The minimum required thickness of the real surface can be determined, by a skilled person, based on manufacturability, stress conditions and features of the material.

In a particularly interesting application, the reactor of the invention is used for the synthesis of ammonia or methanol.

Preferred configurations include: an inter-bed heat exchanger which is used to cool down the hot effluent from a catalytic bed prior to a subsequent catalytic bed; a pre-heater which is arranged to pre-heat a syngas directed to the catalytic bed; a steam superheater arranged to generate steam above its saturation temperature by exchanging heat with a hot reacted gas generated within the chemical reactor.

A further application of the invention is a revamping procedure wherein a conventional heat exchanger internal to a chemical reactor, such as a shell-and-tube heat exchanger, is replaced by installing a heat exchanger comprising a structure made by additive manufacturing, which is a triply periodic minimal surfaces (TPMS) structure, and a set of boundary elements as above described.

In a further aspect of the invention, the heat exchanger, in addition to said TPMS structure, includes at least one collector portion and a transition zone between the TPMS structure and said collector portion. In the transition zone, the shape of the TPMS structure is continuously modified to the shape of the collector portion.

The transition zone begins at a first location and ends at a second location according to a local coordinate. Conventionally, it is useful to assume that the transition zone begins at a first coordinate z=0 and ends at a second coordinate z=L, wherein said coordinate z is taken along a central main axis of the heat exchanger. Preferably said axis is a main axis of the chemical reactor, typically a vertical central axis in a vertical reactor or a horizontal central axis in a horizontal reactor. Said axis may be an axis of radial symmetry. See for example the axis A-A of FIG. 9 or of FIG. 10.

In the above-defined transition zone, the shape of the heat exchanger evolves gradually from the TPMS structure to the shape of the collector portion. Preferably the transition zone is described mathematically by the equation:

$${G ( x , y , z , S , k )}^{⋆} w 1 ( z ) + {C ( x , y , z , R , t )}^{⋆} w 2 ( z ) = 0 \qquad ( EQ1 )$$

where:
the equation G (x, y, z, S, k)=0 is the above-mentioned equation that describes the TPMS structure,
the equation C (x, y, z, R, t)=0 describes said collector portion,
R is a set of one or more parameters defining the shape of the collector in the equation C,
t is a parameter defining the thickness of the collector,
x, y, and z are, as defined above, spatial coordinates,
w1(z) and w2(z) are weight functions that satisfy the following conditions:

$$w 1 ( 0 ) = 1 and w 1 ( L ) = 0 ;$$

$$w 2 ( 0 ) = 0 and w 2 ( L ) = 1 ;$$

$$0 < w 1 ( z ) < 1 and 0 < w 2 ( z ) < 1 for any z the range 0 < z < L .$$

The above equations of the TPMS structure and the collector portion are indicated with the short notation G=0 and C=0.

The TPMS structure (or TPMS lattice) is described mathematically by the equation G=0, meaning the points whose spatial coordinates x, y and z satisfy the equation G=0 are all and the only points which belong to the TPMS surface. Similarly, the equation C=0 describes said collector portion in the sense that points with coordinates x, y, z to satisfy the equation are the points of the collector portion.

The set R may include one or more parameters according to the shape of the collector. For example, in a simple embodiment the collector is a cylindrical pipe and R is the average radius of the cylindrical pipe. In other embodiments the collector has a more elaborate shape and R contains the parameters necessary to define said shape. For example, R may contain two parameters to define a pipe with elliptical cross section.

Said functions w1(z) and w2(z) are continuous in the z-range of 0 to L. More preferably one or both functions w1 and w2 are monotonic in the range 0<z<L. In accordance with the boundary values at z=0 and z=L, the monotonic property results in said function w1(z) being monotonically decreasing in the range 0<z<L and said function w2(z) being monotonically increasing in the same range.

In a preferred embodiment both the weight functions w1 and w2 are linear functions. An example of said functions w1 and w2 being linear is an embodiment wherein:

$$w 1 ( z ) = 1 - z / L ;$$

$$w 2 ( z ) = z / L .$$

Linearity of the weight functions is generally preferred to provide a gradual transition between the connector and the TPSM structure; however, linearity is not a requisite and in other embodiments said functions w1 and/or w2 may be non-linear functions such as quadratic or of a higher degree with respect to the variable z or periodic such as sinusoidal.

The TPMS defined by the above-mentioned equation G=0 is preferably selected from the group of gyroid surface, Schwarz minimal surface and Neovius surface. More preferably said structure defined by the equation G=0 is a gyroid surface.

In a particularly preferred embodiment, the reactor of the invention includes concentric pipes connected with the TPMS structure of the heat exchanger in the manner described above. More in detail, in this embodiment the reactor has a first pipe and a second pipe wherein the first pipe is coaxial with the second pipe; the heat exchanger is connected to said pipes, preferably by welding; the pipe interface is connected to the heat exchanger core (with TPMS lattice) by a transition region described by said equation EQ1 according to a local coordinate system.

Particularly, the first interface has a first transition region with the TPMS structure whereas the second interface has a second transition region with the TPMS. The first transition region and the second transition region may be located at different locations and consequently the first transition region may be described with the equation EQ1 using a first coordinate system and the second transition region may be described with the same equation EQ1 but using a second coordinate system. The first and second coordinate system may have a different origin, so for example the first transition region is described by equation EQ1 using a coordinate z1 and the second transition region is described using a second coordinate z2. The extension of the transition regions may be different, so the first transition region may extend from z1=0 to z1=L1 and the second transition region may extend from z2=0 to z2=L2.

A further aspect of the invention is modularity of the TPMS-based heat exchanger. Modularity is advantageous when a desired size of the heat exchanger is bigger than optimum size for additive manufacturing.

A compact heat exchanger according to the present invention may replace a heat exchanger of rod baffle type. A rod-baffle heat exchanger typically has a multitude of pipes whose length is about 4 to 10 times the diameter of the tubesheet, meaning that length is the predominant size of the exchanger. A TPMS-based compact heat exchanger, in some cases, may not have the same longitudinal extension because pressure drops would be excessive and/or the length may excess the manufacturing capabilities.

The applicant has found, particularly for the service as interchanger, that a preferred shape of a compact exchanger according to the present invention is cylindrical with a height about 1 to 4 times the diameter. Longer height will result in a higher pressure drop, while a larger diameter could lead to a lower Reynold number, therefore reducing the effectiveness of the heat exchanger.

A possible solution to overcome the previous constraint is presented. The solution includes moving the interchanger below the bed and making the heat exchanger modular. The heat exchanger can be composed of many pieces welded together at the lateral linear boundaries. A related advantage is the possible shortening of the bed in the axial direction, and therefore shortening of the pressure vessel.

### DESCRIPTION OF THE FIGURES

The invention is further elucidated with the following figures:

FIG. 1 illustrates a heat exchanger body according to an embodiment of the invention,

FIG. 2 illustrates a boundary element that can be used with the heat exchanger body of FIG. 1,

FIG. 3 illustrates the heat exchanger body of FIG. 1 with the boundary element of FIG. 2,

FIG. 4 is a scheme of a sectional view of a catalytic reactor according to an embodiment,

FIG. 5 compares a sectional view of a catalytic reactor according to an embodiment of the prior art and of a catalytic reactor according to an embodiment of the invention,

FIGS. 6 to 11 illustrate further embodiments of the invention, as detailed below.

The following terminology can be used for a better understanding of the invention.

The TPMS lattice denotes a lattice structure which divides a region of space into two separate sub-portions. Each of the sub-portions, in operation, can be traversed by a heat exchange medium.

The term core section denotes the heat exchanger including the TPMS lattice and at least one shell. In a common embodiment, the core section includes the TPMS lattice and two coaxial shells, namely an outer shell and an inner shell. The outer shell and inner shell are preferably cylindrical.

The term transition lattice may be used to denote the transition portion described by the above-mentioned equation EQ1.

The term transition section denotes the portion of the heat exchanger formed by the transition section and the one or more shell(s) of the heat exchanger.

The term collectors denotes the piping element feeding a fluid to the heat exchanger, or collecting a fluid from the heat exchanger.

The heat exchanger is formed essentially by the core section and one or two transition sections. Preferably, the assembly of the core section and any transition section is printed monolithically as a single piece.

The term interface denotes the connection between the transition section and the collectors. At the interface, the heat exchanger is connected to the collectors. The connection may be made by welding or another technique such as a flange.

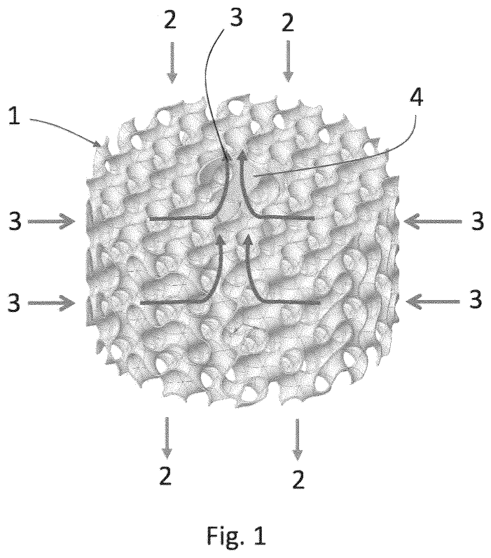

Turning to the figures, FIG. 1 illustrates a TPMS lattice 1 and an inner shell 4. The lattice 1 is of the gyroid type according to a preferred embodiment.

The lattice 1 is traversed axially by a first fluid 2 and radially by a second fluid 3. For example, the fluid 2 is effluent of a catalytic bed upstream and the fluid 3 is fresh gas to be preheated. The example illustrates an inward radial flow but in other embodiment an outward radial flow can be provided.

The streams of fluids 2 and 3 are directed to different channels of the structure of the body 1. The inner shell 4 integrally formed in the body 1 collects the radial flow of the fluid 3.

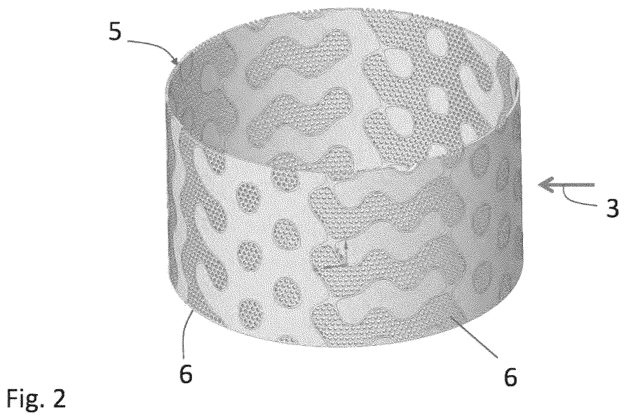

FIG. 2 illustrates a boundary element in the form of an outer shell 5. Said outer shell 5 has a cylindrical surface with a pattern of gas-permeable regions 6. Said gas-permeable regions 6 match the inlets of one side of the body 1, for example the side to be traversed by the radial flow of the fluid 3. The gas-permeable regions 6 have a pattern of small holes for retaining a granular catalyst.

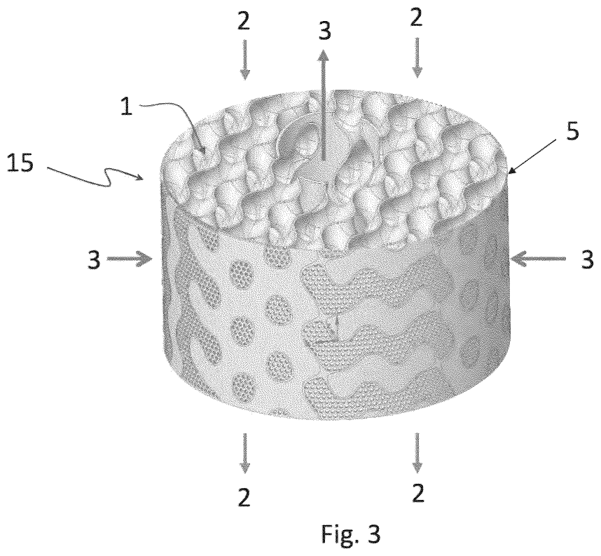

FIG. 3 illustrates a core section of a heat exchanger 15 formed by the lattice 1, the inner shell 4 and the outer shell 5. In this example, the fluid 3 is distributed by the outer shell 5 into one side of the lattice 1 and, after passage through the lattice, said fluid 3 is collected by the inner shell 4. Similarly, a suitable distributor and collector can be provided for the axial flow 2.

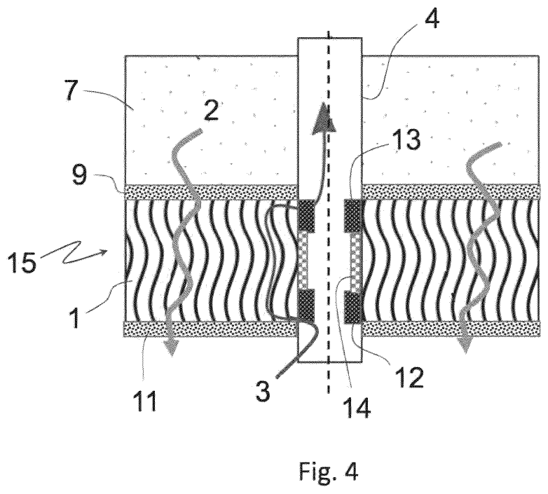

FIG. 4 shows a catalytic reactor 10 containing a heat exchanger 15 according to an embodiment of the invention. Particularly, FIG. 4 discloses a possible arrangement of a heat exchanger according to the invention located below a catalytic bed.

FIG. 4 shows the following items:
Heat exchanger 15
TPMS lattice 1
Catalytic bed 7
Inner collector 4
Inlet plate 9
Outlet plate 11
Inlet ring 12
Outlet ring 13
Sealing ring 14.

The catalytic bed 7 is arranged internally in a catalytic reactor. The heat exchanger 15 is arranged below the catalytic bed 7 and is delimited on the central-bottom side by the inlet ring 12 and by the outlet ring 13 located on the central-top portion of the heat exchanger.

The inlet ring 12 and the outlet ring 13 are part of a set of boundary elements together with a sealing ring 14 arranged in between the two rings 12 and 13. The set of boundary elements also includes an inlet plate 9 and an outlet plate 11 respectively arranged above and below the heat exchanger 15.

The inlet ring 12 and outlet ring 13 have a pattern of apertures arranged to match the first side of the lattice 1 whilst the inlet plate 9 and outlet plate 11 have a pattern of apertures arranged to match the second side of the lattice 1.

A reagent gas mixture (not shown) is conveyed to the catalytic bed where it reacts over the catalyst to generate the hot effluent 2. The hot effluent 2 crosses axially the inlet plate 9 and enters the first side of the heat exchanger body 1 wherein it indirectly exchanges heat with a cold stream 3 that traverses the second side of the heat exchanger body 1. After exchanging heat with the cold stream 3, the reaction effluent 2 leaves the heat exchanger via the outlet plate 11.

The cold stream 3 enters the second side of the heat exchanger 15 via the inlet ring 12, and after exchanging heat with the hot effluent 2 returns to the inner collector via the outlet ring 13.

The sealing ring 14 prevents the cold stream 3 from entering the first side of the heat exchanger body 1 which is traversed by the hot effluent 2.

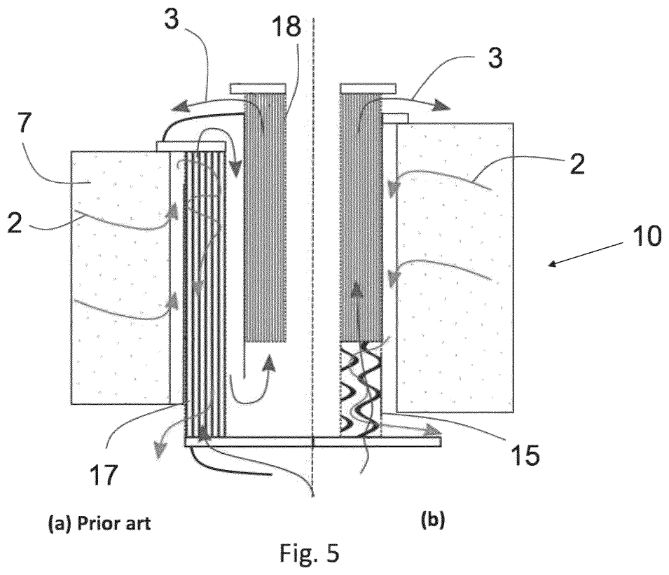

FIG. 5 shows a comparison with the prior art. FIG. 5 (a) illustrates a sectional view of a catalytic reactor according to an embodiment of the prior art and FIG. 5 (b) illustrates a catalytic reactor according to an embodiment of the invention.

The catalytic reactor of the prior art comprises a catalytic bed 7, a shell and tube heat exchanger 17 and a start-up heater 18. As visible in figure, the start-up heater 18 is arranged adjacent (usually concentrically) to the shell and tube heat exchanger 17. Said heat exchanger may be of the rod-baffle type.

In the inventive catalytic reactor 10, the shell and tube heat exchanger 17 is replaced by a heat exchanger 15 made by additive manufacturing and including a TPMS lattice as described above. The heat exchanger 15 provides a greater heat transfer coefficient compared to the shell and tube heat exchanger of the prior art and, for this reason, it can be manufactured with a compact design and arranged below the start-up heater 18 as shown in figure. This arrangement frees space for more catalyst. Advantageously the size of the catalytic bed 7 can be increased bringing enormous advantages to the process such as a higher production rate.

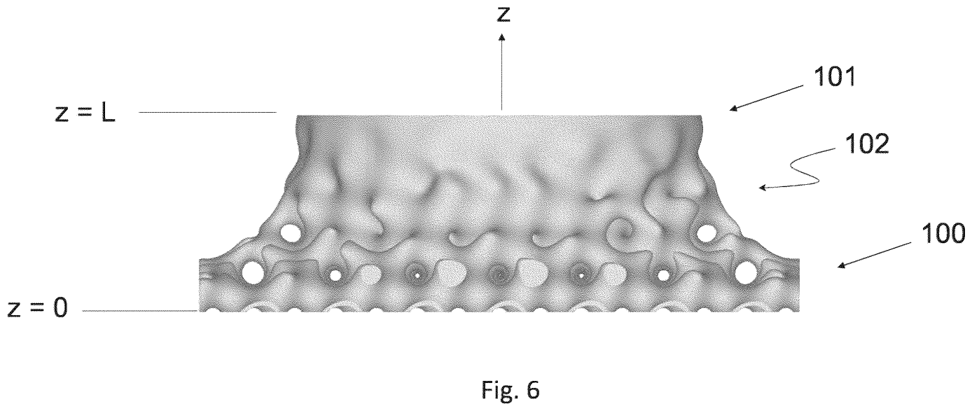

FIG. 6 illustrates an embodiment of transition between a gyroid lattice 100 and a cylinder 101. The transition begins at an axial coordinate z=0 and is completed at an axial coordinate z=L. In the transition region 102, the shape of the structure is described by the equation EQ1 as discussed above. Consequently, the structure evolves from the lattice interface 100 to the circular interface 101.

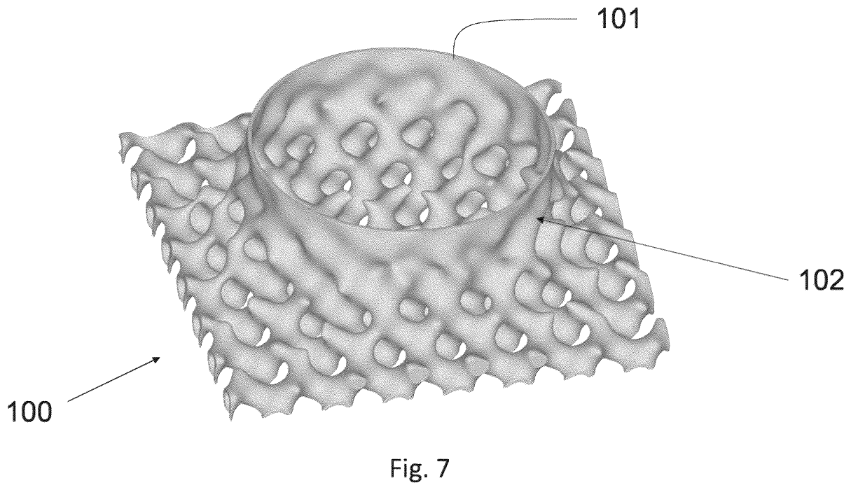

FIG. 7 illustrates the transition of FIG. 6 in a perspective view.

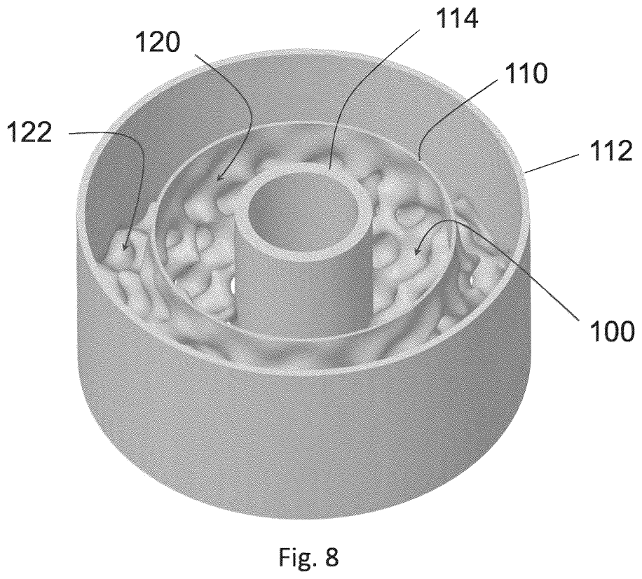

FIG. 8 illustrates an embodiment where a gyroid lattice 100 is contained within an outer tube 112. The lattice 100 has a first transition section 120 forming a pipe 110 for connection with a collector which separates an inlet gas flow from an outlet gas flow. The lattice 100 has also a second transition section 122 with the outer tube 112. A central by-pass pipe 114 is also illustrated. Said bypass pipe 114 passes through the lattice 100 and can be obtained formally by removing a cylinder from the structure of the lattice 100 (Boolean operation).

It can be seen from FIG. 8 that the cylindrical portions 110, 112 represent suitable connection portion with conventional (cylindrical) pipes. The connection portions 110, 112 can be formed by additive manufacturing together with the lattice 100 and can be connected (e.g. welded) to a conventional pipes.

The transition regions 120, 122 can be defined mathematically with equation EQ1, provided that a suitable coordinate z (local coordinate) is adopted.

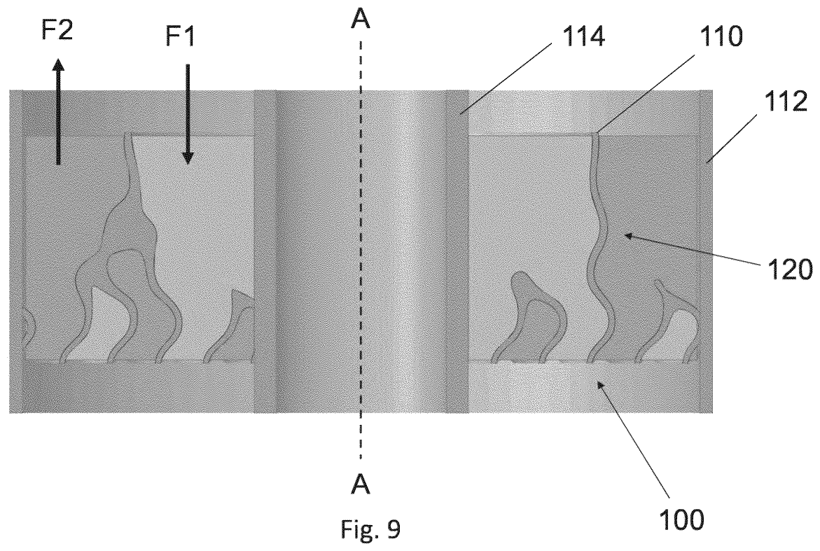

FIG. 9 illustrates a sectional view of the transition between the lattice 100 and the inner ring 110. FIG. 9 illustrates also the input F1 and output F2 of a heat exchange fluid to/from the lattice 100. The transition region 120 is readily appreciated in this figure. FIG. 9 illustrates also the main axis A-A of the heat exchanger.

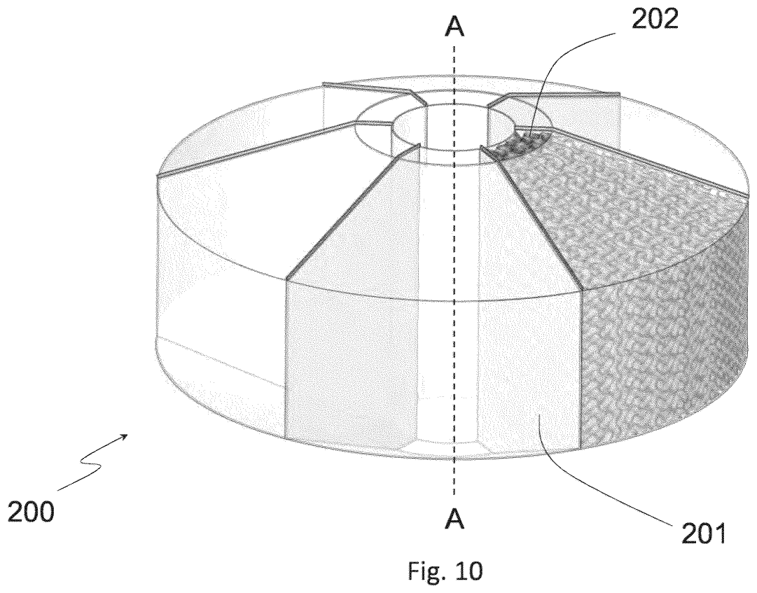

FIG. 10 illustrates a modular heat exchanger according to an embodiment of the invention, wherein a compact heat exchanger 200 includes sectors 201 connected in parallel. If made of a weldable material, the sectors 201 may be welded together to form the exchanger 200. Each sector 201 includes a TPMS structure according to any of the embodiments described above.

FIG. 10 illustrates a preferred shape of the modular heat exchanger 200 which is toroid-shaped. The region 202 close to the main axis A-A is the preferred location for inlets and outlets of both side of the heat exchanger 200, namely the side in communication with the cooling fluid and heating fluid, the other side being closed by the boundary of the pressure vessel.

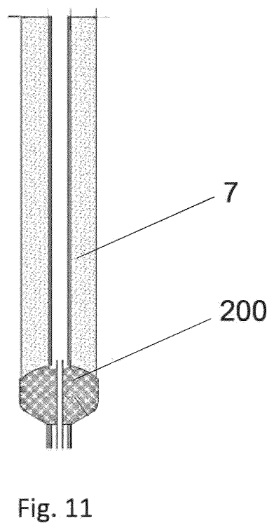

FIG. 11 illustrates the heat exchanger 200 of FIG. 10 positioned below a catalytic bed 7. FIG. 11 allows to appreciate the compactness of the heat exchanger 200 compared to a conventional longitudinally-extended rod-baffle exchanger.

1-23. (canceled)

24. A chemical reactor, comprising:
a catalytic bed adapted to perform a catalytic reaction;
a heat exchanger arranged to transfer heat from a first fluid, which is an effluent of the catalytic bed, to a second fluid, which is a heating or cooling medium, wherein the heat exchanger comprises a heat-exchange structure made by additive manufacturing wherein the structure is a triply periodic minimal surface (TPMS); and
a set of boundary elements configured to distribute and collect the first fluid and second fluid to/from the TPMS structure;
wherein the TPMS structure has a first side and a second side;
wherein the first side defines a first path that can be traversed by a heat exchange medium,
wherein the second side defines a second path that can be traversed by a heat exchange medium,
wherein the first path is separated from the second path, so that a medium traversing the TPMS structure through the first path does not come into direct contact with a medium in the second path,
wherein the set of boundary elements being configured so that the effluent is distributed into and collected from the first side only, and the heating or cooling medium is distributed into and collected from the second side only, so that the effluent can traverse the TPMS structure through the first path and the heating or cooling medium can traverse the TPMS structure through the second path, to obtain indirect transfer of heat from the effluent to the cooling medium or from the heating medium to the effluent.

25. The chemical reactor according to claim 24 wherein the heat exchanger includes:
at least one connection portion having a suitable shape for connection with a collector or distributor pipe; and
a transition zone between the TPMS structure and the connection portion;
wherein the TPMS structure and the connection portion are made monolithically as a single piece by additive manufacturing;
wherein, in the transition zone, the shape of the TPMS structure continuously change its shape to the shape of the connection portion.

26. The chemical reactor according to claim 25 wherein the transition zone extends, according to a coordinate z along an axis of the heat exchanger, from a starting location at z=0 to an end location at z=L;
where, in the above-defined transition zone, the shape of the heat exchanger is described mathematically by the following equation:
$${G ( x , y , z , S , k )}^{*} w 1 ( z ) + {C ( x , y , z , R , t )}^{*} w 2 ( z ) = 0 \qquad ( EQ1 )$$
where:
the equation G (x, y, z, S, k)=0 describes the TPMS structure,
the equation C (x, y, z, R, t)=0 describes the connection portion,
S is a set of one or more parameters defining the shape of the TPMS structure,
k is a parameter defining the thickness of the TPMS structure,
R is a set of parameters defining the shape of the connection portion,
t is a parameter defining the thickness of the connection portion,
x, y, and z are spatial coordinates,
w1(z) and w2(z) are weight functions that satisfy the following conditions:
$$i ) w 1 ( 0 ) = 1 and w 1 ( L ) = 0 ;$$
$$ii ) w 2 ( 0 ) = 0 and w 2 ( L ) = 1 ;$$
$$iii ) 0 < w 1 ( z ) < 1 and 0 < w 2 ( z ) < 1 for any z the range 0 < z < L .$$

27. The chemical reactor according to claim 26 wherein the functions w1(z) and w2(z) are continuous and monotonic in the range 0<z<L.

28. The chemical reactor according to claim 27 wherein the functions are linear, wherein the functions are w1(z)=1−z/L and w2(z)=z/L.

29. The chemical reactor according to claim 26 wherein the heat exchanger includes a first connection portion and a second connection portion integrally formed with the TPMS structure for connection with two coaxial pipes, each connection portion being connected to the heat exchanger by a respective transition region described by the equation (EQ1) according to a local coordinate system.

30. The chemical reactor according to claim 24 wherein the set of boundary elements includes one or more surface elements having a gas-permeable pattern arranged to match a pattern of inputs or outputs in the TPMS structure.

31. The chemical reactor according to claim 24 wherein the TPMS structure has an annular shape and one side of the structure is traversed radially, and the set of boundary elements includes an inner cylindrical shell and/or an outer cylindrical shell, to distribute and/or collect the effluent or the cooling medium to/from the radially traversed side of the TPMS structure.

32. The chemical reactor according to claim 31, wherein the catalytic bed has an annular shape and is arranged around the TPMS structure and the outer cylindrical shell acts as a boundary element for the TPMS structure and as a retainer wall of catalyst granules which form the catalytic bed.

33. The chemical reactor according to claim 24, wherein the TPMS structure is placed under the catalytic bed and is arranged to support the weight of the catalytic bed.

34. The chemical reactor according to claim 24 wherein both the catalytic bed and the TPMS structure have an annular shape, the TMPS structure being arranged coaxially outside or inside the catalytic bed.

35. The chemical reactor according to claim 24, wherein:
the TPMS structure has an annular shape;
the TPMS structure is traversed axially by both fluids; and
the boundary elements include a first ring-shaped boundary element (9) placed above the structure and a second ring-shaped element below the structure, to distribute and collect the fluid which traversed the structure in the axial direction.

36. The chemical reactor according to claim 24, wherein the TPMS structure has an annular shape, and the set of boundary elements includes a first element extending over a portion of an inner surface or outer surface of the TPMS structure, and a second element arranged around a different portion of the surface, so that one fluid enter the TPMS structure and leaves the TPMS structure through different portions of the surface.

37. The chemical reactor according to claim 24, wherein the TPMS structure is selected from the group of Gyroid surface, Schwarz minimal surface, Neovius surface.

38. The chemical reactor according to claim 24, wherein the heat exchanger including the TPMS structure, the boundary elements and transitions is made of Inconel.

39. The chemical reactor according to claim 24, wherein the additive manufacturing technique is any of the following: powder bed fusion, such as any of direct metal laser sintering, electron beam melting, selective heat sintering, selective laser melting and selective laser sintering; binder jetting; stereolithography; fused deposition modelling; digital light process; multi jet fusion; polyjet; or direct energy deposition.

40. The chemical reactor according to claim 24, wherein the chemical reactor is configured for the synthesis for ammonia or methanol.

41. The chemical reactor according to claim 24, wherein the heat exchanger is an inter-bed heat exchanger, a pre-heater, a steam superheater, or combinations thereof.

42. A method of using of the chemical reactor according to claim 24 for the synthesis of ammonia or for the synthesis of methanol.

43. A method of designing a chemical reactor, including a provision of a heat exchanger located internally in the chemical reactor, wherein the heat exchanger includes a triply periodic minimal surface (TPMS) lattice structure that defines two separate heat exchange sides to be traversed by a first heat exchange medium and a second heat exchange medium; the chemical reactor further including at least one connection portion for connection with a distribution pipe or collector pipe of a heat exchange medium to/from one side of the heat exchanger, the method comprising:
modelling of a transition zone for connection between the lattice structure and the connection portion, according to the following equation EQ1:
$${G ( x , y , z , S , k )}^{*} w 1 ( z ) + {C ( x , y , z , R , t )}^{*} w 2 ( z ) = 0 \qquad ( EQ1 )$$
where:
the equation G (x, y, z, S, k)=0 describes the TPMS lattice structure,
the equation C (x, y, z, R, t)=0 describes the collector portion,
S is a set of one or more parameters defining the shape of the TPMS lattice,
R is a set of one or more parameters defining the shape of the connection portion,
k is a parameter defining the thickness of the TPMS structure,
t is a parameter defining the thickness of the collector,
x, y, and z are spatial coordinates,
the transition zone is assumed to begin at z=0 and to end at z=L;
w1(z) and w2(z) are weight functions that satisfy the following conditions:
$$w 1 ( 0 ) = 1 and w 1 ( L ) = 0 ;$$
$$w 2 ( 0 ) = 0 and w 2 ( L ) = 1 ;$$
$$0 < w 1 ( z ) < 1 and 0 < w 2 ( z ) < 1 for any z the range 0 < z < L .$$

44. The method according to claim 43 wherein the functions w1(z) and w2(z) are continuous and monotonic in the range 0<z<L.

45. The method according to claim 44 wherein the functions are linear, wherein preferably the functions are w1(z)=1−z/L and w2(z)=z/L.

46. The method according to claim 43, wherein the reactor is a reactor for the synthesis of ammonia or methanol.
